(19) United States
(12) Patent Application Publication
(10) Pub. No.: US 2013/0156884 A1
(43) Pub. Date: Jun. 20, 2013

(54) PROTEASE ENZYMES FOR INCREASED PROTEIN DIGESTION RATE AND ABSORPTION AND METHODS OF USING THE SAME

(71) Applicant: Mark L. Anderson, Glenwood, NJ (US)

(72) Inventor: Mark L. Anderson, Glenwood, NJ (US)

(73) Assignee: TRIARCO INDUSTRIES, Wayne, NJ (US)

(21) Appl. No.: 13/329,861

(22) Filed: Dec. 19, 2011

(51) Int. Cl. C12N 9/50; C12N 9/62; A23L 1/305; C12N 9/54

(52) U.S. Cl. 426/2; 435/221; 435/225; 426/63

(57) ABSTRACT

A food supplement comprises at least one protease enzyme selected from (i) CAS #9001-92-7, IUB 3.4.23.18 protease from Aspergillus; (ii) CAS #9001-92-7, IUB 3.4.21.7 protease from Bacillus; (iii) CAS #9001-61-0, IUB 3.4.11.1 protease from Aspergillus; (iv) CAS #9074-07-1, IUB 3.4.21.63 protease from Aspergillus; (v) CAS #9073-78-3, IUB 3.4.24.27 protease from Aspergillus; (vi) CAS #9025-49-4, IUB 3.4.23.18 protease from Aspergillus; and (vii) combinations thereof. The food supplement may further comprise a protein, a stabilizer, a matrix modifier, a carrier, a preservative, a bulking agent, a dessicant, an emulsifier, an enzyme coating, or combinations thereof. Disclosed is a method of increasing protein absorption in the gastrointestinal system of a human being comprising the step of ingesting the food supplement as identified above.

### BACKGROUND OF THE INVENTION

1. Field of the Invention

This invention relates to a food supplement comprised of at least one fungal or bacterial protease enzyme, which exhibits increased proteolytic activity producing increased protein digestion rate and absorption in the presence of pepsin, and methods of using the same.

2. Background

Protease enzymes isolated from the fermentation of various strains of fungi and bacteria have been used as additives in the food processing industry for almost a century (Underkofler, L. A., et al., “Microbiological process report—Production of microbial enzymes and their applications,” Applied Microbiology, Vol. 6, pp. 212-221 (1958)). Enzymes have also been added to animal feed to induce weight gain in various farm animals used for food and dairy production (Leahy K T, et al., “Effects of treating corn silage with alpha amylase and(or) sorbic acid on beef cattle growth and carcass characteristics,” J. Anim. Sci. 1990; 68(2):490-7, and Stokes, M R., “Effects of an enzyme mixture, an inoculant, and their interaction on silage fermentation and dairy production,” J. Dairy Sci. 1992; 75(3):764-73). More recently, enzymes have been promoted in the dietary supplement industry in the category of digestive aids. Sales of digestive aids and enzymes grew over 8% in 2009 from the previous year, with enzymes accounting for $69 million of this growing category (Wright, R., The Enzyme Market, Nutraceuticals World (June 2010)).

There is a variety of different protease enzymes produced from various sources or even the same source but by different methods that are used as dietary supplement digestive aids. However, they are generally used and added without evaluating how they may function under the specified conditions of use.

One reason for this lack of understanding may be that there is no accepted, uniform analytical methodology available for comparing the relative activities of different proteases from the same or different sources as digestive aids. There is also no accepted, uniform analytical methodology available for evaluating their relative activities under conditions of intended use which is in the presence of endogenous digestive enzymes and for the purpose of determining an effective dose.

The Food and Chemical Codex (FCC) and the United States Pharmacopeia Dietary Supplement Compendium (USP) list the same assays that differentiate protease activity between fungal sources and bacterial sources. However, the assays are done under different conditions and each report different protease units. No explanation is offered as to how these enzyme units differ quantitatively, how the protease activity will be affected when used as a digestive aid or if the protease activity is affected in the presence of endogenous digestive enzymes. For example, using FCC or USP methodology, the activity of a neutral fungal protease produced from Aspergillus oryzae may be reported in HUT units (from hemoglobin assayed at pH 4.7 and 40° C.), while acid proteases activity from the same source is reported in SAP units (from casein assayed at pH 3 and 37° C.). No explanation is offered as to how these enzyme units relate to each other quantitatively, how the protease activity of either will be affected when used as a digestive aid or if the protease activity is affected in the presence of endogenous digestive enzymes. Another example is the activity of two bacterial proteases. One from Bacillus subtilis and one from Bacillus lichentformis which are both reported in PC units (from casein at pH 7.0 and 37° C.) using the same FCC and USP methodology. No information or explanation is offered as to how or if the protease activity of either will be affected when used as a digestive aid or if the protease activity is affected in the presence of endogenous digestive enzymes. Also, the precise differences between HUT, SAP and PC units have not generally been quantified in the art.

Variations in substrate, pH and temperature used in compendial activity assays for analysis make it difficult to compare activities of the various protease enzymes. Also, none of these assays provide a mechanism to determine if a particular enzyme, enzyme combination or enzyme formula will contribute to increasing the rate of protein digestion in the presence of pepsin and pancreatin. “Contribute to digestive activity” as used herein refers to increasing the rate of digestion above the rate produced by pepsin or pancreatin, either separately or combined, on specific substrates under physiological conditions. This information would be valuable in determining differences in digestive activity among proteases as well as an effective dose of protease enzyme to use as a dietary supplement to increase the rate of protein digestion and absorption. Increasing the rate of postprandial protein digestion may decrease indigestion and facilitate rapid increase of peptide and amino acid levels in the blood. In turn, rapid hyperaminoacidemia helps increase muscle mass by increasing protein absorption, which is valuable for athletes, as well as those suffering from age related protein loss and, possibly, sarcopenia. (Boirie, Y. et al., “Slow and fast dietary proteins differently modulate postprandial protein accretion,” Proc. Natl. Acad. Sci. USA 1997; 94; 14930-14935; Fujita, S. et al., “Branched-Chain Amino Acids Metabolism, Physiological Function, and Application,” J. Nutr. 2006; 136: 277S-280S; and Dangin et al., “Influence of the Protein Digestion Rate on Protein Turnover in Young and Elderly Subjects,” J. Nutr. 2002; 132: 3228S-32335).

To address the problems in the art, as disclosed herein, various protease enzymes have been evaluated for proteolytic activity for use as dietary supplements herein and analyzed for contribution to the digestive activity of specific proteins in the presence of pepsin. Upon ingestion, the protease enzymes in the present food supplement are combined and interact with the ingested proteins and enzymes naturally present in the human stomach such as pepsin. Pepsin is released by the cells in the stomach in the form of pepsinogen and must be activated to pepsin at a pH of below 5. The activated pepsin then degrades food proteins into peptides. Of the enzymes present in the stomach, pepsin is understood to be the most efficient in cleaving peptide bonds between hydrophobic and preferably aromatic amino acids.

Proteolytic activity is evaluated and analyzed herein by developing and implementing an analytical method capable of standardizing and comparing proteolytic activity under physiological conditions of temperature, pH and the presence of endogenous digestive enzymes such as pepsin. The present disclosure solves the problems identified in the art above and identifies specific protease enzymes which, in the presence of pepsin, increase proteolytic activity under gastric digestive conditions leading to enhanced rate of protein absorption (digestion) and an improved food supplement.

### SUMMARY OF THE INVENTION

The present disclosure is directed to a food supplement comprising at least one protease enzyme selected from the group consisting of: (i) CAS #9001-92-7, IUB 3.4.23.18 protease from Aspergillus; (ii) CAS #9001-92-7, IUB 3.4.21.7 protease from Bacillus; (iii) CAS #9001-61-0, IUB 3.4.11.1 protease from Aspergillus; (iv) CAS #9074-07-1, IUB 3.4.21.63 protease from Aspergillus; (v) CAS #9073-78-3, IUB 3.4.24.27 protease from Aspergillus; (vi) CAS #9025-49-4, IUB 3.4.23.18 protease from Aspergillus: and (vii) combinations thereof. In certain embodiments, CAS #9001-92-7, IUB 3.4.23.18 protease from Aspergillus is from the species oryzae; CAS #9001-92-7, IUB 3.4.21.7 protease from Bacillus is from the species subtilis; CAS #9001-61-0, IUB 3.4.11.1 protease from Aspergillus is from the species oryzae; CAS #9074-07-1. IUB 3.4.21.63 protease from Aspergillus is from the species melleus; CAS #9073-78-3, IUB 3.4.24.27 protease from Aspergillus is from the species oryzae; and CAS #9025-49-4, IUB 3.4.23.18 protease from Aspergillus is from the species niger. In preferred embodiments, the at least one protease enzyme is a combination of (i) CAS #9001-92-7, IUB 3.4.23.18 protease from Aspergillus oryzae and (iv) CAS #9074-07-1, IUB 3.4.21.63 protease from Aspergillus melleus, a combination of (i) CAS #9001-92-7, IUB 3.4.23.18 protease from Aspergillus oryzae and (v) CAS #9073-78-3, IUB 3.4.24.27 protease from Aspergillus oryzae, or a combination of (iv) CAS #9074-07-1, IUB 3.4.21.63 protease from Aspergillus melleus and (v) CAS #9073-78-3, IUB 3.4.24.27 protease from Aspergillus oryzae. In certain embodiments, the food supplement further comprises a protein selected from whey, soy, casein, and combinations thereof, or further comprises an additional ingredient selected from stabilizer, a matrix modifier, a carrier, a preservative, a bulking agent, a dessicant, an emulsifier, an enzyme coating, and combinations thereof. In a certain embodiment, the matrix modifier is selected from salts of dihydrogen phosphate, salts of nitric acid, salts of citric acid, salts of polynicotinic acid, salts of picolinic acid, weak organic acids, potyphenolic compounds with at least one pka of 5 or less, and combinations thereof.

The present invention is also directed to a method of increasing protein absorption (digestion rate) in the gastrointestinal system of a human being comprising the step of ingesting a food supplement comprised of at least one protease enzyme selected from the group consisting of: (i) CAS #9001-92-7, IUB 3.4.23.18 protease from Aspergillus; (ii) CAS #9001-92-7, IUB 3.4.21.7 protease from Bacillus; (iii) CAS #9001-61-0, IUB 3.4.11.1 protease from Aspergillus; (iv) CAS #9074-07-1, IUB 3.4.21.63 protease from Aspergillus; (v) CAS #9073-78-3, IUB 3.4.24.27 protease from Aspergillus: (vi) CAS #9025-49-4, IUB 3.4.23.18 protease from Aspergillus; and (vii) combinations thereof. In preferred embodiments of the method of increasing protein absorption in the gastrointestinal system of a human being, the at least one protease enzyme is a combination of (i) CAS #9001-92-7, IUB 3.4.23.18 protease from Aspergillus oryzae and (iv) CAS #9074-07-1, IUB 3.4.21.63 protease from Aspergillus melleus, a combination of (i) CAS #9001-92-7, IUB 3.4.23.18 protease from Aspergillus oryzae and (v) CAS #9073-78-3, IUB 3.4.24.27 protease from Aspergillus oryzae, or a combination of (iv) CAS #9074-07-1, IUB 3.4.21.63 protease from Aspergillus melleus and (v) CAS #9073-78-3, IUB 3.4.24.27 protease from Aspergillus oryzae. In a certain embodiment, the inventive method further comprises ingesting a protein selected from the group consisting of whey, soy, casein, and combinations thereof.

### BRIEF DESCRIPTION OF THE FIGURE

FIG. 1 shows the integral area of the relative increase in free amine concentration at specific time points as a product of proteolytic activity in simulated gastric fluid (SGF) incubated at 37° C. Graphed results include: analysis of whey protein incubated in SGF and pepsin (WPC in SGF-WP); Protease Enzyme #4 combined with whey protein and incubated in SGF without pepsin (Enzyme #4+WPC in SGF-NP) and Protease Enzyme #4 combined with whey protein and incubated in SGF with pepsin (Enzyme #4+WPC in SGF-WP). The absorbance values at time zero (t=0) were subtracted from each time point in each respective data set.

FIG. 2 shows the raw absorbance data for Protease Enzyme #4, which was used to calculate the integral area of the relative increase in free amine concentration at specific time points as shown in FIG. 2.

FIG. 3 shows the integral area of the relative increase in free amine concentration at specific time points as a product of proteolytic activity in simulated gastric fluid (SGF) incubated at 37° C. Graphed results include: analysis of whey protein incubated in SGF and pepsin (WPC in SGF-WP); Protease Enzyme #1 combined with whey protein and incubated in SGF without pepsin (Enzyme #1+WPC in SGF-NP) and Protease Enzyme #1 combined with whey protein and incubated in SGF with pepsin (Enzyme #1+WPC in SGF-WP). The absorbance values at time zero (t=0) were subtracted from each time point in each respective data set.

FIG. 4 shows the integral area of the relative increase in free amine concentration at specific time points as a product of proteolytic activity in simulated gastric fluid (SGF) incubated at 37° C. Graphed results include: analysis of whey protein incubated in SGF and pepsin (WPC in SGF-WP); Protease Enzyme #2 combined with whey protein and incubated in SGF without pepsin (Enzyme #2+WPC in SGF-NP) and Protease Enzyme #2 combined with whey protein and incubated in SGF with pepsin (Enzyme #2+WPC in SGF-WP). The absorbance values at time zero (t 0) were subtracted from each time point in each respective data set.

FIG. 5 shows the integral area of the relative increase in free amine concentration at specific time points as a product of proteolytic activity in simulated gastric fluid (SGF) incubated at 37° C. Graphed results include: analysis of whey protein incubated in SGF and pepsin (WPC in SGF-WP); Protease Enzyme #3 combined with whey protein and incubated in SGF without pepsin (Enzyme #3+WPC in SGF-NP) and Protease Enzyme #3 combined with whey protein and incubated in SGF with pepsin (Enzyme #3+WPC in SGF-WP). The absorbance values at time zero (t=0) were subtracted from each time point in each respective data set.

FIG. 6 shows the integral area of the relative increase in free amine concentration at specific time points as a product of proteolytic activity in simulated gastric fluid (SGF) incubated at 37° C. Graphed results include: analysis of whey protein incubated in SGF and pepsin (WPC in SGF-WP); Protease Enzyme #5 combined with whey protein and incubated in SGF without pepsin (Enzyme #5+WPC in SGF-NP) and Protease Enzyme #5 combined with whey protein and incubated in SGF with pepsin (Enzyme #5+WPC in SGF-WP). The absorbance values at time zero (t) were subtracted from each time point in each respective data set.

FIG. 7 shows the integral area of the relative increase in free amine concentration at specific time points as a product of proteolytic activity in simulated gastric fluid (SGF) incubated at 37° C. Graphed results include: analysis of whey protein incubated in SGF and pepsin (WPC in SGF-WP); Protease Enzyme #6 combined with whey protein and incubated in SGF without pepsin (Enzyme #6+WPC in SGF-NP) and Protease Enzyme #6 combined with whey protein and incubated in SGF with pepsin (Enzyme #6+WPC in SGF-WP). The absorbance values at time zero (t=0) were subtracted from each time point in each respective data set.

### DETAILED DESCRIPTION OF THE INVENTION

The objective of this invention was to determine and teach the enzyme or enzyme combination that could most rapidly digest protein and promote protein absorption in the gastrointestinal system. The protease enzymes tested are among the several classes of fungal and bacterial enzymes accepted for possible use in dietary supplements. The inventors discovered that the proteolytic activity of protease enzymes can vary greatly depending on the protein substrate and the presence of pepsin and simulated gastric fluid. The inventors also discovered that protease activity under these conditions is not obvious and cannot be accurately deduced from the activity specifications provided from the manufacturer. Also, no data or information was heretofore found comparing proteolytic activity on varying dietary protein substrates in the presence of simulated gastric fluid and in the presence and absence of pepsin.

The first embodiment of the present invention is directed to a food supplement comprising at least one protease enzyme selected from the following specified by CAS number, IUB number and genus: (i) CAS #9001-92-7, IUB 3.4.23.18 protease from Aspergillus; (ii) CAS #9001-92-7, IUB 3.4.21.7 protease from Bacillus; (iii) CAS #9001-61-0, IUB 3.4.11.1 protease from Aspergillus; (iv) CAS #9074-07-1, IUB 3.4.21.63 protease from Aspergillus; (v) CAS #9073-78-3, IUB 3.4.24.27 protease from Aspergillus; (vi) CAS #9025-49-4, IUB 3.4.23.18 protease from Aspergillus; and (vii) combinations thereof.

A protease enzyme is defined herein as an enzyme, which is derived from a fungal or bacterial source, and is capable of breaking down proteins and their degradation products, polypeptides and peptides, by hydrolysis and is active in a pH environment ranging from a pH of about 2 to a pH of about 8. “Protease enzyme” and “proteolytic enzyme” are used interchangeably herein. All protease enzymes suitable for use in the present invention are commercially available from known sources or can be derived by known methods.

Preferably. CAS #9001-92-7, IUB 3.4.23.18 protease from Aspergillus may be from the species oryzae; CAS #9001-92-7, IUB 3.4.21.7 protease from Bacillus may be from the species subtilis; CAS #9001-61-0, IUB 3.4.11.1 protease from Aspergillus may be from the species oryzae; CAS #9074-07-1, IUB 3.4.21.63 protease from Aspergillus may be from the species melleus; CAS #9073-78-3, IUB 3.4.24.27 protease from Aspergillus may be from the species oryzae; and CAS #9025-49-4, IUB 3.4.23.18 protease from Aspergillus may be from the species niger.

The detailed manufacturer specifications of these enzymes are as follows. CAS #9001-92-7, IUB 3.4.23.18 protease from Aspergillus oryzae, referred to hereinafter as “Protease Enzyme #1,” has an active pH range of 6-9, an optimal pH of 7.5, an active temperature range of 25-60° C., an optimal temperature of 50° C., and an activity assay of 400,000 HU/g. Protease Enzyme #1 is light brown to brown in color and has a loss on drying (“LOD”) of less than 10%.

CAS #9001-92-7, TUB 3.4.21.7 protease from Bacillus subtilis, referred to hereinafter as “Protease Enzyme #2,” has an active pH range of 6-8, an optimal pH of 7.5, an active temperature range of 40-60° C., an optimal temperature of 55° C., and an activity assay of 2,000,000 PC/g. Protease Enzyme #2 is light tan to tan in color and has an LOD of less than 10%.

CAS #9001-61-0, IUB 3.4.11.1 protease from Aspergillus oryzae, referred to hereinafter as “Protease Enzyme #3,” has an active pH range of 5.5-8.5, an optimal pH of 7, an active temperature range of 30-60° C., an optimal temperature of 50° C., and an activity assay of 500 LAP/g. Protease Enzyme #3 is light brown to brown in color and has an LOD of less than 10%.

CAS #9074-07-1, IUB 3.4.21.63 protease from Aspergillus melleus, referred to hereinafter as “Protease Enzyme #4,” has an active pH range of 5-11, an optimal pH of 8, an active temperature range of 30-50° C., an optimal temperature of 45° C., and an activity assay of 203,000 HUT/g.

CAS #9073-78-3, IUB 3.4.24.27 protease from Aspergillus oryzae, referred to hereinafter as “Protease Enzyme #5,” has an active pH range of 3-11, an optimal pH of 7, an active temperature range of 25-60° C., an optimal temperature of 45° C., and an activity-assay of 657,000 HUT/g.

CAS #9025-49-4, IUB 3.4.23.18 protease from Aspergillus niger, referred to hereinafter as “Protease Enzyme #6,” has an active pH range of 1-4, an optimal pH of 2.5, an active temperature range of 30-60° C., an optimal temperature of 55° C., and an activity assay of 111,000 HUT/g.

Activity assay as used herein refers to the data published in the Food and Chemical Codex (FCC), United States Pharmacopeia Dietary Supplement Compendium (USP) or obtained from a reputable vender (i.e., Bio-Cat, Troy, Va.) to determine the activity of fungal or bacterial protease enzymes. A standard value of 7500 HUT units of activity per gram of substrate was chosen for comparison of relative enzyme activities. The activity of Protease Enzymes #4, #5 and #6 were purchased with the activity expressed in HUT units. Protease Enzymes #1, #2 and #3 were purchased in HU, PC and LAP units, respectively. An approximate conversion of activity units for these enzymes to HUT units was mathematically estimated using product weight equivalence and molar equivalence. The amount of enzyme/g substrate used in the assay was calculated from the enzyme activity reported on the certificate of analysis (C of A).

One HUT is defined as the amount of enzyme that produces free, liberated tyrosine having the same absorbance value as 1.10 mcg/mL of tyrosine/minute. For Protease Enzyme #4 the C of A reported an activity of 203,000 HUT/g, 7500 HUT/g=36.9 mg added/g of substrate. For Protease Enzyme #5 the C of A reported an activity of 657,000 HUT/g, 7500 HUT/g=11.4 mg to be added/g of substrate. For Protease Enzyme #6 the C of A reported an activity of 111,000 HUT/g, 7500 HUT/g=67.6 mg to be added/g of substrate.

One Hemoglobin Unit (HU) is the amount of enzyme which will liberate 447 mcg of non-protein nitrogen in 30 minutes=14.9 mcg nitrogen/minute. Based upon product weight equivalence, this is approximately 13.54 times more product than 1 HUT (1 HU=13.54 HUT and 553.9 HU=7500 HUT). For Protease Enzyme #1 the C of A reported an activity of 400,000 HU/g, 553.9 HU=1.38 mg (7500 HUT) to be added/g of substrate.

One PC is defined as the amount of enzyme that produces the equivalent of 1.5 mcg/mL of tyrosine/min. Based upon product weight equivalence, this is approximately 36.4% more product than 1 HUT (1 PC=1.364 HUT and 5498.53 PC=7500 HUT). For Protease Enzyme #2 the C of A reported an activity of 2,000,000 PC/g, 5498.53 PC=2.75 mg (7500 HUT) to be added/g of substrate.

One Leucine Aminopeptidase Activity Unit (LAP) is defined as the amount of enzyme required to liberate 1 mcmol of leucine/minute from leucine p-nitroanilide and based on a 1:1 molar ratio calculated from p-nitroaniline. One mcmole of pnitroaniline=138.12 mcg and, based upon product weight equivalence, this is approximately 125.56 times more product than 1 HUT (1 LAP=125.56 HUT and 59.7 LAP=7500 HUT). For Protease Enzyme #3 the C of A reported an activity of 500 LAP/g, 59.7 LAP=119.4 mg (7500 HUT) to be added/g of substrate.

The food supplement of the first embodiment may preferably be designed to be ingested with protein in order to convert the ingested dietary protein into free amino acids in the gastrointestinal system, which then may be used by the body for muscle protein synthesis. Any protein is suitable for use with the present invention, with foods having a higher protein content by weight being more preferable. Preferred proteins include food products derived from animal protein, plant protein isolate and plant protein concentrate (e.g., soy, oat, wheat, rice, pea, corn and rapeseed protein isolate/concentrate) and milk protein (e.g., casein and whey protein). In a preferred embodiment, the protein is whey, soy or casein. In a certain embodiment, about 1 to about 500 mg of protease enzyme in the form of a tablet, capsule or separate product may be ingested per about 25 g protein ingested. In another embodiment of a food or protein supplement, about 1 to about 50 mg protease enzyme may be ingested per gram of protein ingested.

In an embodiment of the present invention, the food supplement further comprises a protein, preferably selected from the group consisting of whey, soy, casein, and combinations thereof. The terms whey, soy and casein encompass all forms, types and concentrations of whey, soy and casein protein commercially available and utilized in food supplements. Whey and “whey protein”, soy and “soy protein”, and casein and “casein protein” are used interchangeably herein. One of ordinary skill in the art would readily understand how to determine the amount of protein to be added to the food supplement.

In a certain preferred embodiment, the food supplement contains: (i) CAS #9001-92-7, IUB 3.4.23.18 protease from Aspergillus oryzae; (ii) CAS #9001-92-7, IUB 3.4.21.7 protease from Bacillus subtilis; (iii) CAS #9001-61-0, IUB 3.4.11.1 protease from Aspergillus oryzae; (iv) CAS #9074-07-1, IUB 3.4.21.63 protease from Aspergillus melleus; or (v) CAS #9073-78-3, IUB 3.4.24.27 protease from Aspergillus oryzae; and whey or soy.

In an embodiment of the present invention, the food supplement further comprises an additional ingredient selected from a stabilizer, a matrix modifier, a carrier, a preservative, a sweetener, a bulking agent, a binding agent, a dessicant, a lubricating agent, a filler, a solubilizing agent, an emulsifier, an enzyme coating, including, but not limited to, time release coatings, and combinations thereof. Examples of bulking agents suitable for use in the present invention include, but are not limited to, gum acacia, gum Arabic, xanthan gum, guar gum, pectin, and combinations thereof. Examples of sweeteners suitable for use in the present invention include, but are not limited to, glucose, fructose, stevia, acesulfame potassium, erythritol, and combinations thereof. Examples of coatings suitable for use in the present invention include, but are not limited to, ethyl cellulose, hydroxypropyl methyl cellulose, shellac, and combinations thereof. Examples of preservatives suitable for use in the present invention include, but are not limited to, antimicrobial preservatives, such as benzoic acid, benzyl alcohol, calcium acetate, and combinations thereof. Examples of binding agents suitable for use in the present invention include, but are not limited to, croscarmellose sodium, povidone, dextrin, and combinations thereof. Examples of dessicants suitable for use in the present invention include, but are not limited to, silicon dioxide, calcium silicate, and combinations thereof. Examples of lubricating agents suitable for use in the present invention include, but are not limited to, magnesium stearate, stearic acid, silicon dioxide, and combinations thereof. Examples of fillers suitable for use in the present invention include, but are not limited to, maltodextrin, dextrin, starch, calcium salts, and combinations thereof. Examples of solubilizing agents suitable for use in the present invention include, but are not limited to, cyclodextrin, lecithin, and combinations thereof. Examples of emulsifiers suitable for use in the present invention include, but are not limited to, vegetable oils, fatty acids and mono-, di- and triglycerides, such as medium chain triglycerides or their esters. Any of the additional ingredients may be present in an amount of about 0.5% to about 95% of the food supplement. One of ordinary skill in the art would readily understand how to determine the amount of additional ingredient to be added to the food supplement.

Any commercially acceptable stabilizer known to be suitable for use in food products may be used in the present invention. Suitable stabilizers include, but are not limited to, Agar, pectin and lecithin.

Any commercially acceptable matrix modifier with a buffering capacity between pH 1 and pH 6 known to be suitable for use in food products, including monoprotic and polyprotic weak organic acids and inorganic acids with at least one pKa value of 5 or less and a pH of between 1 and 6, may be used in the present invention. Preferable matrix modifiers include flavonoids, flavonols, isoflavones, catechins, gallic acid, salts of monohydrate or dihydrate phosphates, sulfates, ascorbates, amino acids, sodium citrate, citric acid including citrate salts, benzoates, gluconic acid including gluconic acid salts, acetic acid including acetate salts, picolinic acid salts, nicotinic acid salts, phenolic or polyphenolic compounds with at least one pKa value of 5 or less and a pH between pH 1 and pH 6.0, as well as combinations thereof. Other suitable buffers include dicalcium phosphate, sodium phosphate, potassium phosphate and the like. In a preferred embodiment, the matrix modifier is selected from salts of dihydrogen phosphate (such as ammonium dihydrogen phosphate), salts of nitric acid (such as magnesium nitrate), salts of citric acid (such as calcium citrate), salts of polynicotinic acid (such as chromium (III) polynicotinate), salts of picolinic acid (such as chromium picolinate), weak organic acids, polyphenolic compounds with at least one pka of 5 or less, and combinations thereof. More preferably, the matrix modifier is 10% calcium citrate or 0.3% chromium (III) polynicotinate. Both citric acid and nicotinic acid are weak organic acids with some buffering capacity, which is a quantitative measure of the resistance of a buffer solution to pH change. These acids can tolerate increasing or decreasing levels of excreted stomach acid, which may help the activation of pepsinogen to pepsin. It may also help maintain a buffered environment to preserve the activity of pepsin and supplemental enzymes.

In a certain embodiment of the present invention, the food supplement contains: a combination of (i) CAS #9001-92-7, IUB 3.4.23.18 protease from Aspergillus oryzae and (iv) CAS #9074-07-1, IUB 3.4.21.63 protease from Aspergillus melleus; whey or casein; and calcium citrate or chromium (III) polynicotinate. In another embodiment, the food supplement contains: a combination of (i) CAS #9001-92-7, IUB 3.4.23.18 protease from Aspergillus oryzae and (v) CAS #9073-78-3, IUB 3.4.24.27 protease from Aspergillus oryzae; whey or casein; and calcium citrate or chromium (III) polynicotinate. In another embodiment, the food supplement contains: a combination of (iv) CAS #9074-07-1, IUB 3.4.21.63 protease from Aspergillus melleus and (v) CAS #9073-78-3. IUB 3.4.24.27 protease from Aspergillus oryzae; whey, soy, casein, or a combination thereof; and calcium citrate or chromium (III) polynicotinate.

Any commercially acceptable carrier known to be suitable for use in food products may be used in the present invention. Preferable carriers include maltodextrin, polypropylene, starch, modified starch, gum, proteins, amino acids, as well as mixtures thereof. Depending on the type of carrier used, the food supplement of the present invention may be in any form known in the art, including but not limited to a powder, capsule, tablet, or liquid formulation. The term food supplement, as used herein, also encompasses nutritional supplements, dietary supplements, weight-gain supplements, weight loss supplements, recovery supplements, sports nutrition supplements, digestive aid supplements, pancreatic enzyme replacement supplements, and the like.

The food supplement of the present invention, upon ingestion by a human, increases the rate of protein digestion and absorption in the gastrointestinal system. This is particularly true in the presence of pepsin. As used herein, proteolytic activity refers to the total integrated area of the enzyme-protein digestion graph reported in millions of units (M), which is a quantitative representation of the increase in peptides and amino acids produced over time by the digestion (breakdown of protein to its component amino acids and peptides) of protein. Proteolytic activity is directly related to the amount of protein digested and ultimately absorbed in the gastrointestinal system in the form of peptides and amino acids. The higher the proteolytic activity, the greater the rate of protein digestion and absorption in the gastrointestinal system. Digestion of protein is necessary because undigested protein is too large to traverse the intestinal cells and tissue. Increasing the rate of digestion increases the amount of amino acids and peptides available for absorption from the stomach and small intestine before reaching the caecum and being fermented in the large intestine. When the rate of protein absorption is increased, higher blood levels of amino acids are attained for muscle protein synthesis to increase muscle mass. Boirie, Y., et al.; “Slow and fast dietary proteins differently modulate postprandial protein accretion,” Proc. Natl. Acad. Sci. USA. Vol. 94, pp. 14930-14935 (1997). Protein absorption and protein digestion are used interchangeably herein.

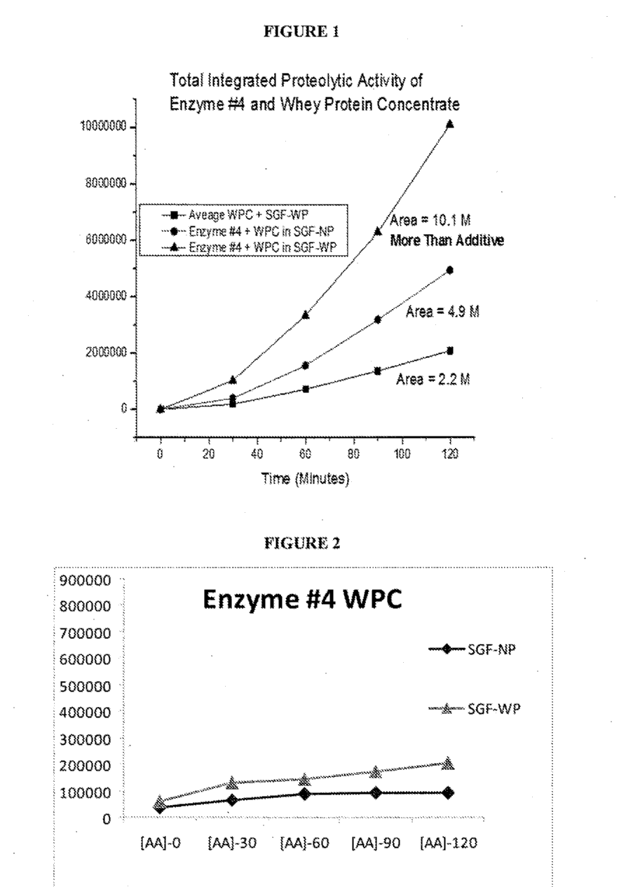

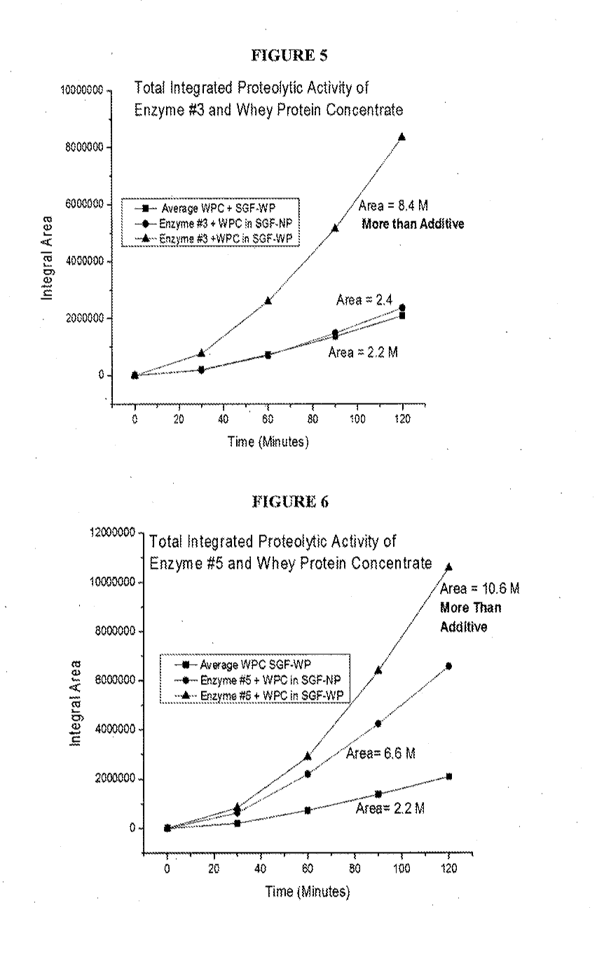

Proteolytic activity as disclosed herein is understood to be determined under gastric digestive conditions with and without pepsin, unless indicated otherwise. Gastric digestive conditions are approximated by mixing a portion of each protein and protein-enzyme dry blend (100 mg) with 1 mL simulated gastric fluid (pH 1.2) made to USP specifications without pepsin (SGF-NP) and mixing a portion of each protein and protein-enzyme dry blend with simulated gastric fluid made to USP specifications (SGF-USP) containing 3.2 mg purified pepsin per mL (2,566 U/mL) at a pH of about 1.2 (USP 30, page 810). All protein-SGF mixtures had a pH of about 3.5. All mixtures were incubated in a 37° C. water bath until assayed at times 0, 30, 60, 90 and 120 minutes as shown in FIGS. 1, and 3-7. As noted above, the increased proteolytic activity caused by the present food supplement can be with respect to protein consumed independently by a human, with respect to protein contained within the food supplement, or both.

It would be expected that, since the optimal pH and temperature ranges of Protease Enzymes #1-#5 are very similar, proteolytic activity would be similar for the protease enzymes in the presence of pepsin with the same proteins. Protease Enzyme #6 has a very low optimal pH and a similar optimal temperature range as Protease Enzymes #1-#5. The rate of proteolytic activity in gastric fluid for Protease Enzyme #6 would be expected to be higher than for Protease Enzymes #1-#5, because of its low optimal pH since the rate of enzyme activity is a function of pH and temperature. However, the proteolytic activity of the protease enzymes varies in the presence of pepsin. In a preferred embodiment, the proteolytic activity of each of Protease Enzymes #1-#5 in the presence of pepsin is greater than the proteolytic activity of the protease enzyme in the absence of pepsin. It is believed that the pepsin and protease enzymes act together to increase the overall proteolytic activity in the gastrointestinal system upon ingestion. In addition, in a preferred embodiment, the proteolytic activity of Protease Enzymes #1-#5 in the presence of pepsin is greater than the proteolytic activity of pepsin in the absence of the protease enzyme. In a preferred embodiment, with whey protein or soy protein, the proteolytic activity of Protease Enzymes #1-#5 in the presence of pepsin is greater than the addition of the proteolytic activity of the protease enzyme in the absence of pepsin plus the proteolytic activity of pepsin in the absence of the protease enzyme (“a greater than additive effect”).

Preferably, proteolytic activity of Protease Enzyme #1 in the presence of pepsin increases about 1% to about 10%, more preferably about 5%, with whey protein, and about 30% to about 39%, more preferably about 34%, with soy protein, over the sum of the proteolytic activity of the protease enzyme in the absence of pepsin plus the proteolytic activity of pepsin absent the protease enzyme.

Preferably, proteolytic activity of Protease Enzyme #2 in the presence of pepsin increases about 75% to about 85%, more preferably about 80%, with whey protein, and about 130% to about 140%, more preferably about 135%, with soy protein, over the sum of the proteolytic activity of the protease enzyme in the absence of pepsin plus the proteolytic activity of pepsin absent the protease enzyme.

Preferably, proteolytic activity of Protease Enzyme #3 in the presence of pepsin increases about 78% to about 88%, more preferably about 83%, with whey protein, and about 45% to about 55%, more preferably about 50%, with soy protein, over the sum of the proteolytic activity of the protease enzyme in the absence of pepsin plus the proteolytic activity of pepsin absent the protease enzyme.

Preferably, proteolytic activity of Protease Enzyme #4 in the presence of pepsin increases about 38% to about 47%, more preferably about 42%, with whey protein, and about 8% to about 17%, more preferably about 12%, with soy protein, over the sum of the proteolytic activity of the protease enzyme in the absence of pepsin plus the proteolytic activity of pepsin absent the protease enzyme.

Preferably, proteolytic activity of Protease Enzyme #5 in the presence of pepsin increases about 15% to about 25%, more preferably about 20%, with whey protein, and about 11% to about 21%, more preferably about 16%, with soy protein, over the sum of the proteolytic activity of the protease enzyme in the absence of pepsin plus the proteolytic activity of pepsin absent the protease enzyme.

TABLE 1
The percent increase in proteolytic activity in preferred
embodiments of the protease enzyme in the presence of pepsin
over the addition of proteolytic activity of the protease
enzyme in the absence of pepsin plus the proteolytic activity
of pepsin absent the protease enzyme is shown below.

| | Whey Protein | Soy Protein |
|---|---|---|
| Protease Enzyme #1 | about 5% | about 34% |
| Protease Enzyme #2 | about 80% | about 135% |
| Protease Enzyme #3 | about 83% | about 50% |
| Protease Enzyme #4 | about 42% | about 12% |
| Protease Enzyme #5 | about 20% | about 16% |

In a certain embodiment, the at least one protease enzyme in the food supplement is a combination of Protease Enzyme #1 and Protease Enzyme #4 (“Protease Enzyme #1+#4”) and, in another embodiment, the food supplement further contains a protein selected from the group consisting of whey, soy, casein and combinations thereof. In a preferred embodiment thereof, the proteolytic activity of the combination of Protease Enzyme #1 and Protease Enzyme #4 in the absence of pepsin with whey protein is about 40% to about 48%, more preferably about 44%, greater than the sum of the proteolytic activity of Protease Enzyme #1 in the absence of pepsin plus proteolytic activity of Protease Enzyme #4 in the absence of pepsin, both with whey protein. In a preferred embodiment, the proteolytic activity of the combination of Protease Enzyme #1 and Protease Enzyme #4 has a greater than additive effect with whey and soy protein in the presence of pepsin. In a preferred embodiment thereof, the proteolytic activity of the combination of Protease Enzyme #1 and Protease Enzyme #4 in the presence of pepsin with whey protein is about 18% to about 28%, more preferably about 23%, greater than the sum of the proteolytic activity of Protease Enzyme #1 in the absence of pepsin plus activity Protease Enzyme #4 in the absence of pepsin plus the proteolytic activity of pepsin absent the protease enzyme. With soy protein, the proteolytic activity of the combination of Protease Enzyme #1 and Protease Enzyme #4 in the presence of pepsin is about 10% to about 18%, more preferably about 14%, greater than the sum of the proteolytic activity of Protease Enzyme #1 in the absence of pepsin plus the proteolytic activity of Protease Enzyme #4 in the absence of pepsin plus the proteolytic activity of pepsin absent the protease enzymes. With casein, the proteolytic activity of the combination of Protease Enzyme #1 and Protease Enzyme #4 in the presence of pepsin is about 10% to about 18%, more preferably about 13%, greater than the sum of the proteolytic activity of Protease Enzyme #1 in the absence of pepsin plus the proteolytic activity of Protease Enzyme #4 in the absence of pepsin plus the proteolytic activity of pepsin absent the protease enzymes.

In a certain embodiment, the at least one protease enzyme in the food supplement is a combination of Protease Enzyme #1 and Protease Enzyme #5 (“Protease Enzyme #1+#5”), and, in another embodiment, the food supplement further contains a protein selected from the group consisting of whey, soy, casein and combinations thereof; in a preferred embodiment, the protein is whey. In a preferred embodiment, the proteolytic activity of the combination of Protease Enzyme #1 and Protease Enzyme #5 has a greater than additive effect with whey protein in the absence and in the presence of pepsin. In a preferred embodiment thereof, the proteolytic activity of the combination of Protease Enzyme #1 and Protease Enzyme #5 in the absence of pepsin with whey protein is about 4% to about 13%, more preferably about 7%, greater than the sum of the proteolytic activity of Protease Enzyme #1 in the absence of pepsin plus the proteolytic activity of Protease Enzyme #5 in the absence of pepsin, both with whey protein. In a preferred embodiment thereof, the proteolytic activity of the combination of Protease Enzyme #1 and Protease Enzyme #5 in the presence of pepsin with whey protein is about 8% to about 16%, more preferably about 11% to about 12%, greater than the sum of the proteolytic activity of Protease Enzyme #1 in the absence of pepsin plus the proteolytic activity Protease Enzyme #5 in the absence of pepsin plus the proteolytic activity of pepsin absent the protease enzymes.

In a certain embodiment, the at least one protease enzyme in the food supplement is a combination of Protease Enzyme #4 and Protease Enzyme #5 (“Protease Enzyme #4+#5”) and in another embodiment, the food supplement further contains a protein selected from the group consisting of whey, soy, casein and combinations thereof; in a preferred embodiment, the protein is whey. In a preferred embodiment thereof, the proteolytic activity of the combination of Protease Enzyme #4 and Protease Enzyme #5 in the absence of pepsin with whey protein is about 33% to about 43%, more preferably about 38%, greater than the sum of the proteolytic activity of Protease Enzyme #4 in the absence of pepsin plus the proteolytic activity of Protease Enzyme #5 in the absence of pepsin, both with whey protein. In a preferred embodiment thereof, the proteolytic activity of the combination of Protease Enzyme #4 and Protease Enzyme #5 in the presence of pepsin with whey protein is about 34% to about 44%, more preferably about 39%, greater than the sum of the proteolytic activity of Protease Enzyme #4 in the absence of pepsin plus the proteolytic activity of Protease Enzyme #5 in the absence of pepsin plus the proteolytic activity of pepsin absent the protease enzymes.

In one embodiment of the invention, the proteolytic activity of Protease Enzyme #1+#4, Protease Enzyme #1+#5 or Protease Enzyme #4+#5 in the presence of pepsin with whey protein is increased, with the addition of 10% calcium citrate (matrix modifier) (weight/weight) to the food supplement, by about 5% to about 20%, and more preferably about 8% to about 18%; and in the presence of pepsin with casein, proteolytic activity is increased by about 70% to about 980%, and more preferably about 80% to about 920%. In a preferred embodiment, the proteolytic activity of the combination of Protease Enzyme #1+#4 in the presence of pepsin with whey protein is increased with the addition of 10% calcium citrate to the food supplement by about 17%; and in the presence of pepsin with casein, proteolytic activity is increased by about 88%. In another preferred embodiment, the proteolytic activity of the combination of Protease Enzyme #1+#5 in the presence of pepsin with whey protein is increased with the addition of 10% calcium citrate to the food supplement by about 9%; and in the presence of pepsin with casein, proteolytic activity is increased by about 295%. In a further preferred embodiment, the proteolytic activity of the combination of Protease Enzyme #4+#5 in the presence of pepsin with whey protein is increased with the addition of 10% calcium citrate to the food supplement by about 9%; in the presence of pepsin with soy protein, proteolytic activity is increased by about 2%; and in the presence of pepsin with casein, proteolytic activity is increased by about 911%. Preferably, the matrix modifier may be present in an amount sufficient to maintain a pH below 5 or a pH suitable to activate pepsinogen. This pH may also be maintained by adding a mixture of weak acids. For example, if chromium picolinate is used as the matrix modifier, one could use a mixture of citrate and chromium picolinate to obtain a suitable formulation comprising enzyme proteases and protein to also deliver an active amount of Cr. Using chromium picolinate alone to maintain a pH below 5 may result in a toxic amount of chromium.

In other embodiments of the invention, with the addition of 0.3% chromium (III) polynicotinate (a matrix modifier) to the food supplement, the proteolytic activity with casein protein in the presence of pepsin with Protease Enzyme #1+#4 is increased by about 37%, with Protease Enzyme #1+#5 is increased by about 343%, and with Protease Enzyme #4+#5 is increased by about 678%.

The second embodiment of the invention is directed to a method of increasing protein absorption in the gastrointestinal system of a human being comprising the step of ingesting a food supplement comprising at least one protease enzyme selected from the group consisting of (i) Protease Enzyme #1; (ii) Protease Enzyme #2; (iii) Protease Enzyme #3; (iv) Protease Enzyme #4; (v) Protease Enzyme #5; (vi) Protease Enzyme #6; and (vii) combinations thereof. In preferred embodiments of the second embodiment, the at least one protease enzyme is a combination of Protease Enzyme #1 and Protease Enzyme #4, Protease Enzyme #1 and Protease Enzyme #5, or Protease Enzyme #4 and Protease Enzyme #5.

Details regarding increased proteolytic activity and hence increased rate of protein absorption, food supplement composition, and Protease Enzymes #1-#6 and combinations thereof are as set forth above with regard to the first embodiment of the invention. The term ingesting as used herein refers to swallowing, drinking, chewing or the like of the food supplement.

In a preferred embodiment, the method of increasing protein absorption in the gastrointestinal system of a human being further comprises the step of ingesting a protein selected from whey, soy and casein. Protein, whey, soy and casein are defined as above. In a further preferred embodiment thereof, the protein is whey or soy and proteolytic activity increases over about 30% in the presence of pepsin. Protein ingestion may be accomplished by including the protein in the food supplement to be ingested in the first step or by consuming protein separately at the time of ingesting the food supplement, i.e., before, after or simultaneously with ingesting the food supplement.

Specific embodiments of the disclosure will now be demonstrated by reference to the following general methods of manufacture and examples. It should be understood that these examples are disclosed solely by way of illustration and should not be taken in any way to limit the scope of the present disclosure.

### Examples

The proteolytic activity of several fungal and bacterial protease enzymes were compared in gastric digestive conditions with and without pepsin using whey protein concentrate (WPC), soy protein concentrate (SPC) and casein protein concentrate (CSC) as substrates. It is readily understood by one skilled in the art that concentrates will work in the same manner as any form of the same digestible protein. Protease Enzymes #1-#5 were individually dry blended with WPC, SPC or CSC to an equivalent activity level of 7500 HUT/g enzyme reference. Combinations of the protease enzymes were also dry blended with WPC, SPC or CSC to a combined equivalent activity level of 7500 HUT/g. Where necessary, the conversion of enzyme units to HUT units was mathematically estimated using tyrosine molar equivalent ratios, as explained above, and the enzyme activity reported on the certificate of analysis. In addition, dry blends of a reference and the sixth protease enzyme were prepared in the same manner. The enzyme reference was a blend of acid protease from Aspergillus oryzae, CAS #9001-92-7, IUB 3.4.23.18, active pH range 6-9, optimal pH 7.5, active temperature range 25-60° C., optimal temperature 50° C., and acid stable protease from Aspergillus niger, CAS #9025-49-4, IUB 3.4.23.18, active pH range 1-4, optimal pH 2.5, active temperature range 30-60° C., optimal temperature 55° C.

Enzyme activity was determined using a portion of each dry blend (100 mg) mixed with 1 mL simulated gastric fluid made to USP specifications without pepsin (SGF-NP) and simulated gastric fluid made to USP specifications containing 3.2 mg purified pepsin (2,566 U/mL) (SGF-WP). The mixtures were incubated in a 37° C. water bath, and the reaction was stopped utilizing an 80° C. water bath and acid precipitation. The method utilized for quantification of proteolytic activity was a modification of the ninhydrin absorbance assay. Preparation and modification of the ninhydrin absorbance assay is readily understood by one of ordinary skill in the art.

### Sample Preparation

A 100 mg sample of protein or protein enzyme blend (PEB) was added to a test tube containing 1 ml of SGF either with or without pepsin. Immediately upon mixing, a 190 μl aliquot of sample, representing the zero (t0) time point, was transferred to a new test tube and placed in an 80° C. water bath for 30 minutes. The remaining volume of that sample was incubated in a 37° C. water bath. Every 30 minutes for a period of 2 hours, a 190 μl aliquot of sample was transferred into a new test tube and incubated in the 80° C. water bath for 30 minutes. Following incubation, a 2 μl aliquot was transferred into a new micro-centrifuge tube and diluted with 498 μl of deionized water (dH₂O). Also added was 500 μl of 6% trichloroacetic acid (TCA), and the tubes were centrifuged at 16,100 g for 10 minutes to precipitate out and remove large proteins which could interfere with the ninhydrin assay.

### Sample Analysis

Following centrifugation, 500 μl of the sample supernatant (or reference standard solution) was transferred into a fresh glass test tube, and 250 μl of ninhydrin solution (Sigma-Aldrich, Saint Louis, Mo.) was added. The test tubes were mixed by vortex then placed in a 99° C. water bath for 10 minutes to activate the ninhydrin. It was then removed from the water bath and cooled to room temperature before adding 1.25 ml of 95% ethanol and vortex mixing. A 200 μl aliquot of sample was transferred to a well on a microplate, and the absorbance was read at 570 nm in duplicate. The absorbance of a corresponding solute without protein or pepsin was subtracted to account for background absorbance. Quantification of each sample was accomplished using a 5-point, 6-aminocaproic acid standard curve with a linear dynamic range from 10-50 uM. Results were adjusted for dilution and reported in um NH2 equivalents.

The results for each protein in SGF-WP or PEB in either SGF-NP or SGF-WP were graphed versus time after subtracting the value at t0 from each respective time point. The integral area of each graph was then calculated for comparison using OriginPro 8 software. The total integrated area, reported in millions of units (M), was directly proportional to the um NH2 equivalents produced over time and reflects the relative metabolic (proteolytic) activity of the enzymes over the experimental time as shown in FIGS. 1 and 3-7.

FIG. 1 depicts the greater than additive effect on proteolytic activity of Protease Enzyme #4 in the presence of pepsin with whey protein. As shown therein, the integral area of proteolytic activity (“proteolytic activity”) of whey protein under gastric digestive conditions in the presence of pepsin (WPC in SGF-WP) is about 2.2M, proteolytic activity of Protease Enzyme #4 with whey protein under gastric digestive conditions in the absence of pepsin (Enzyme #4 in SGF-NP+WPC) is 4.9M, and proteolytic activity of Protease Enzyme #4 with whey protein under gastric digestive conditions in the presence of pepsin (Enzyme #4 in SGF-WP+WPC) is 10.1M. These results are also tabulated in Table 2 below. The resultant proteolytic activity (10.1M) is greater than the sum of WPC in SGF-WP and Enzyme #4 in SGF-NP+WPC (2.2M+4.9M=7.1M). There is an increase in proteolytic activity of about 44%. Graphed results include analysis of whey protein incubated in SGF and pepsin (WPC in SGF-WP); Protease Enzyme #4 combined with whey protein and incubated in SGF without pepsin (Enzyme #4+WPC in SGF-NP) and Protease Enzyme #4 combined with whey protein and incubated in SGF with pepsin (Enzyme #4+WPC in SGF-WP). The absorbance data for Enzyme #4 is shown in FIG. 2. The raw absorbance data was read at 570 nm after reacting with ninhydrin and indicated an increase in free amine levels for Enzyme #4 over 120 minutes at 37° C. Accordingly, the data in these graphs indicates that a food supplement containing Protease Enzyme #4 with whey protein would increase protein absorption (digestion) upon ingestion.

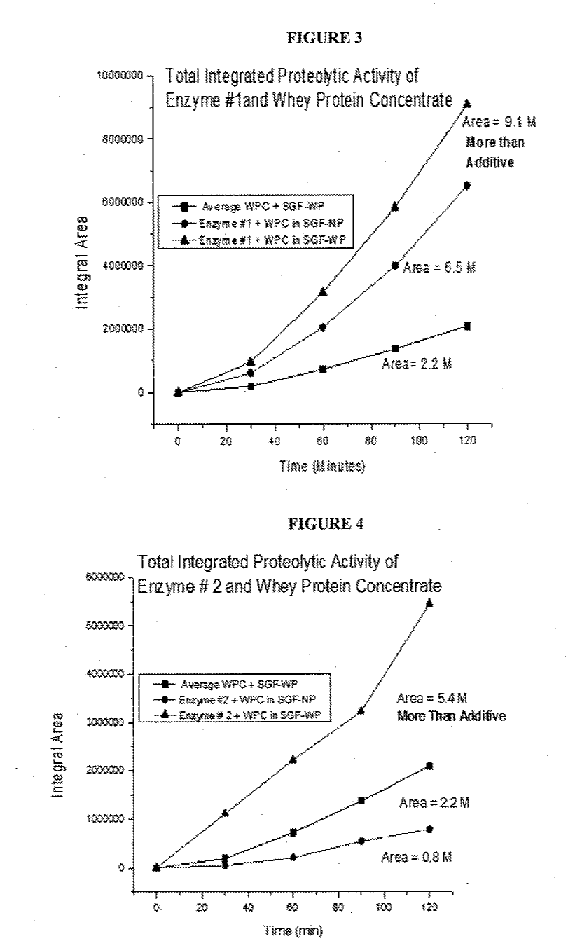

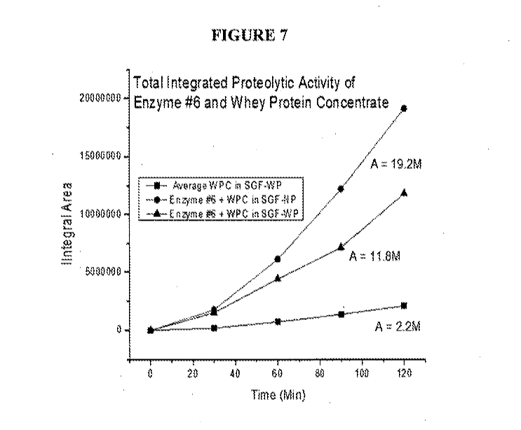

Similarly, FIG. 3 depicts the greater than additive effect on proteolytic activity of Protease Enzyme #1 in the presence of pepsin with whey protein. As shown therein, the integral area of proteolytic activity (“proteolytic activity”) of whey protein under gastric digestive conditions in the presence of pepsin (WPC in SGF-WP) is 2.2M, proteolytic activity of Protease Enzyme #1 with whey protein under gastric digestive conditions in the absence of pepsin (Enzyme #1 in SGF-NP+WPC) is 6.5M, and proteolytic activity of Protease Enzyme #1 with whey protein under gastric digestive conditions in the presence of pepsin (Enzyme #1 in SGF-WP+WPC) is 9.1M. These results are also tabulated in Table 2 below. The resultant proteolytic activity (9.1M) is greater than the sum of WPC in SGF-WP and Enzyme #1 in SGF-NP+WPC (2.2M+6.5M=8.7M). Likewise, FIGS. 4-6 depict the greater than additive effect on proteolytic activity of Protease Enzymes #2, 3 and 5 in the presence of pepsin with whey protein, and FIG. 7 depicts the improved results with Protease Enzyme #6 in the presence of pepsin with whey protein. These graphed results were based on raw absorbance data, as described with respect to Protease Enzyme #4.

### Results

### Individual Enzymes

A comparison of the proteolytic activity of individual enzymes in SGF-NP and the proteolytic activity of pepsin in SGF-WP with WPC, SPC and CPC is shown in Tables 2-4. A comparison of the calculated additive proteolytic activity and measured proteolytic activity of each enzyme in SGF-WP is also shown.

TABLE 2
Proteolytic Activity of Each Protease Enzyme in SGF-NP and SGF-WP with WPC

| | B | C | B + C | D |
|---|---|---|---|---|
| A | Measured proteolytic | Average measured | Calculated additive | Measured proteolytic |
| Protease | activity of enzyme in | proteolytic activity of | proteolytic activity of | activity of enzyme + |
| Enzyme # | SGF-NP on WPC | SGF-WP on WPC | enzyme + SGF-WP on WPC | SGF-WP on WPC |
| 7500 HUT/g | 4.1M | 2.2M | 6.3M | 6.2M |
| Reference | | | | |
| 1 | 6.5M | 2.2M | 8.7M | 9.1M** |
| 2 | 0.8M | 2.2M | 3.0M | 5.4M** |
| 3 | 2.4M | 2.2M | 4.6M | 8.4M** |
| 4 | 4.9M | 2.2M | 7.1M | 10.1M** |
| 5 | 6.6M | 2.2M | 8.8M | 10.6M** |
| 6 | 19.2M | 2.2M | 21.4M | 11.8M |

**Greater than additive.
Column D is the experimentally measured proteolytic activity of each enzyme in SGF-WP on WPC over 2 hours.
Results are reported as total integrated area of each enzyme-substrate graph reported in millions of units (M).

TABLE 3
Proteolytic Activity of Each Protease Enzyme in SGF-NP and SGF-WP with SPC

| | B | C | B + C | D |
|---|---|---|---|---|
| A | Measured proteolytic | Average measured | Calculated additive | Measured proteolytic |
| Protease | activity of enzyme in | proteolytic activity of | proteolytic activity of | activity of enzyme + |
| Enzyme # | SGF-NP on SPC | SGF-WP on SPC | enzyme + SGF-WP on SPC | SGF- WP on SPC |
| 7500 HUT/g | 3.6M | 1.6M | 5.2M | 5.2M |
| Reference | | | | |
| 1 | 8.9M | 1.6M | 10.5M | 14.1M** |
| 2 | 0.7M | 1.6M | 2.3M | 5.4M** |
| 3 | 9.9M | 1.6M | 11.5M | 17.2M** |
| 4 | 10.9M | 1.6M | 12.5M | 14.0M** |
| 5 | 9.7M | 1.6M | 11.3M | 13.1M** |
| 6 | 15.7M | 1.6M | 17.3M | 11.7M |

TABLE 4
Proteolytic Activity of Selected Protease Enzymes in SGF-NP and SGF-WP with CPC

| | B | C | B + C | D |
|---|---|---|---|---|
| A | Measured proteolytic | Average measured | Calculated additive | Measured proteolytic |
| Protease | activity of enzyme in | proteolytic activity of | proteolytic activity of | activity of enzyme + |
| Enzyme # | SGF-NP on CPC | SGF-WP on CPC | enzyme + SGF-WP on CPC | SGF-WP on CPC |
| 1 | 0M | 5.9M | 5.9M | 4.7M |
| 4 | 0M | 5.9M | 5.9M | 3.5M |
| 5 | 0M | 5.9M | 5.9M | 4.6M |

The results reported in Tables 2 and 3 indicate large variations in activity between protease enzymes with both WPC (0.8-6.6 M) and SPC (0.7-10.9M) in the absence of pepsin. As specific examples, Protease Enzymes #1, #2 and #4 have active pH ranges of 6-9, 6-8 and 5-11, respectively, however, they show proteolytic activity with WPC and SPC in the absence of pepsin at pH 3.5, which is the pH of 1 mL of both SGF-NP and SGF-WP containing 100 mg protein (Tables 2 and 3). Since the low end of the active pH range for these enzymes is 5.5 and enzyme activity is a function of pH and temperature, it would have been expected that these enzymes would have no activity at pH 3.5. However, unexpectedly, this was not the case.

As expected, the proteolytic activity of Protease Enzyme #6 is greater than Protease Enzymes #1-#5 in the absence of pepsin (SGF-NP), since it has an optimal pH of 2.5 (active range pH 1-4) and an optimal temperature similar to the others at 55° C. (active range 30-60° C.). However, the data collected in the presence of pepsin, e.g., SGF-WP, demonstrates that, when assayed with pepsin, total calculated proteolytic activity of Protease Enzyme #6 is unexpectedly reduced approximately 38% and 25% using whey and soy, respectively. However, for Protease Enzymes #1-#5, the proteolytic activity increased over the calculated additive proteolytic activity with both whey and soy when assayed with pepsin in SGF-WP (Tables 2 and 3).

### Enzyme Combinations

Comparison of the calculated additive and measured proteolytic activity of specific protease enzyme combinations in SGF-NP and the proteolytic activity of protease enzyme in SGF-WP with WPC, SPC and CPC is shown in Tables 5-7. The calculated additive and measured proteolytic activity of each protease enzyme combination in SGF-WP with WPC, SPC and CPC is also reported.

TABLE 5
Proteolytic Activity of Protease Enzyme Combinations in SGF-NP and SGF-WP with WPC

| | B | | | | |
|---|---|---|---|---|---|
| | Calculated additive | C | D | B + D | E |
| A | proteolytic activity | Measured proteolytic | Average measured | Calculated additive | Measured proteolytic |
| Protease | in SGF-NP on WPC | activity of enzymes in | proteolytic activity of | proteolytic activity of | activity of enzymes + |
| Enzyme # | from Table 2, Col. B | SGF-NP on WPC | SGF-WP on WPC | enzymes + SGF- WP on WPC | SGF-WP on WPC |
| 1 + 4 | 11.4M | 16.4M** | 2.2M | 13.6M | 16.7M** |
| 1 + 5 | 13.1M | 14.0M** | 2.2M | 15.3M | 17.1M** |
| 4 + 5 | 11.5M | 15.9M** | 2.2M | 13.7M | 19.1M** |

TABLE 6
Proteolytic Activity of Protease Enzyme Combinations in SGF-NP and SGF-WP with SPC

| | B | | | | |
|---|---|---|---|---|---|
| | Calculated additive | C | D | B + D | E |
| A | proteolytic activity | Measured proteolytic | Average measured | Calculated additive | Measured proteolytic |
| Protease | in SGF-NP on SPC | activity of enzymes in | proteolytic activity of | proteolytic activity of | activity of enzymes + |
| Enzyme # | from Table 3, Col. B | SGF-NP on SPC | SGF-WP on SPC | enzymes + SGF-WP on SPC | SGF-WP on SPC |
| 1 + 4 | 19.8M | 17.6M | 1.6M | 21.4M | 24.4M** |
| 1 + 5 | 18.6M | 16.9M | 1.6M | 20.2M | 18.9M |
| 4 + 5 | 20.6M | 14.9M | 1.6M | 22.2M | 21M |

TABLE 7
Proteolytic Activity of Protease Enzyme Combinations in SGF-NP and SGF-WP with CPC

| | B | | | | |
|---|---|---|---|---|---|
| | Calculated additive | C | D | B + D | E |
| A | proteolytic activity | Measured proteolytic | Average Measured | Calculated additive | Measured proteolytic |
| Protease | in SGF-NP on CPC | activity of enzymes in | proteolytic activity of | proteolytic activity of | activity of enzymes + |
| Enzyme # | from Table 4, Col. B | SGF-NP on CPC | SGF-WP on CPC | enzymes + SGF-WP on CPC | SGF-WP on CPC |
| 1 + 4 | 0M | 0M | 5.9M | 5.9M | 6.7M** |
| 1 + 5 | 0M | 0M | 5.9M | 5.9M | 2.1M |
| 4 + 5 | 0M | 0M | 5.9M | 5.9M | 0.9M |

Table 5 shows that Protease Enzyme #1+#4. Protease Enzyme #1+#5 and Protease Enzyme #4+#5 combinations assayed with WPC exhibited unexpectedly greater than additive protease activity in SGF-NP and SGF-WP. Similar results are reported with SPC and Protease Enzyme #1+#4 combination in SGF-WP. However, Table 6 shows only Protease Enzyme #1+#4 combination in SGF-WP exhibiting unexpectedly greater than additive protease activity, while the protease activity of Protease Enzyme #1+#5 and Protease Enzyme #4+#5 combinations were less than additive with SPC.

Regarding proteolytic activity with CPC, Table 4 shows that Protease Enzymes #1, #4 and #5, which were most active with WPC and SPC (produced the greatest area under the curve), had no proteolytic activity with CPC in SGF-NP. The data also shows that when the protease enzymes are assayed with pepsin in SGF-WP with CPC, protease activity was slowed compared to SGF-WP with CPC without addition of the enzyme. This is presumably due to the limited solubility of casein in acid. However, surprisingly, Table 7 shows that when the protease enzyme combinations were assayed with pepsin in SGF-WP, Protease Enzyme #1+#4 combination unexpectedly exhibited greater than additive activity, while the other combinations may have slowed protease activity, as in Table 4. Accordingly, Protease Enzyme #1+#5 and Protease Enzyme #4+#5 would reduce protein absorption with casein upon ingestion, while Protease Enzyme #1+#4 combination would unexpectedly result in increased protein absorption of casein.

Enzyme Combinations with Matrix Modifiers

Matrix modifiers chromium (III) polynicotinate (0.3%) and calcium citrate (10%) were combined separately with Protease Enzymes #1, #4 and #5 and WPC, SPC or CPC and assayed in SGF-NP and SGF-WP. Each appropriate combination of matrix modifier (0.3% chromium (III) polynicotinate or 10% calcium citrate) and protease enzyme was dry blended with 100 mg/mL WPC, SPC or CSC and the specified amount of enzyme.

The measured proteolytic activity of specific enzyme combinations with and without the addition of 10% calcium citrate and 0.3% chromium (III) polynicotinate in SGF-NP and SGF-WP with WPC, SPC and CPC is reported in Tables 8-13.

TABLE 8
Proteolytic Activity of Protease Enzyme Combinations
in SGF-NP and Matrix Modifiers with WPC

| | Measured proteolytic | Proteolytic activity of | Proteolytic activity of |
|---|---|---|---|
| Protease | activity of enzymes in | enzymes in SGF-NP + | enzymes in SFG-NP + |
| Enzyme # | SGF-NP from Table 5, Col. C | 10% Ca Citrate | 0.3% Cr Polynicotinate |
| 1 + 4 | 16.4M | 18.2M* | 14.9M |
| 1 + 5 | 14.0M | 17.3M* | 16.6M* |
| 4 + 5 | 15.9M | 20.3M* | 14.7M |

*Proteolytic activity greater than without matrix modifier.

TABLE 9
Proteolytic Activity of Protease Enzyme Combinations
in SGF-WP and Matrix Modifiers with WPC

| | Measured proteolytic | Proteolytic activity of | Proteolytic activity of |
|---|---|---|---|
| Protease | activity of enzymes in | enzymes in SGF-WP + | enzymes in SFG-WP + |
| Enzyme # | SGF-WP from Table 5, Col. E | 1.0% Ca Citrate | 0.3% Cr Polynicotinate |
| 1 + 4 | 16.7M | 19.5M* | 8.4M |
| 1 + 5 | 17.1M | 18.7M* | 8.6M |
| 4 + 5 | 19.1M | 20.8M* | 13.2M |

TABLE 10
Proteolytic Activity of Protease Enzyme Combinations
in SGF-NP and Matrix Modifiers with SPC as Substrate

| | Measured proteolytic | Proteolytic activity of | Proteolytic activity or |
|---|---|---|---|
| Protease | activity of enzymes in | enzymes in SGF-NP + | enzymes in SFG-NP + |
| Enzyme # | SGF-NP from Table 6, Col. C | 10% Ca Citrate | 0.3% Cr Polynicotinate |
| 1 + 4 | 17.6M | 26.5M* | 13.0M |
| 1 + 5 | 16.9M | 23.6M* | 16.3M |
| 4 + 5 | 14.9M | 28.2M* | 13.6M |

TABLE 11
Proteolytic Activity of Protease Enzyme Combinations
in SGF-WP and Matrix Modifiers with SPC

| | Measured proteolytic | Proteolytic activity of | Proteolytic activity of |
|---|---|---|---|
| Protease | activity of enzymes in | enzymes in SGF-WP + | enzymes in SFG-WP + |
| Enzyme # | SGF-WP from Table 6, Col. E | 10% Ca Citrate | 0.3% Cr Polynicotinate |
| 1 + 4 | 24.4M | 20.6M | 10.2M |
| 1 + 5 | 18.9M | 16.3M | 18.6M |
| 4 + 5 | 21M | 21.5M* | 18.1M |

TABLE 12
Proteolytic Activity of Protease Enzyme Combinations
in SGF-NP and Matrix Modifiers with CPC

| | Measured proteolytic | Proteolytic activity of | Proteolytic activity of |
|---|---|---|---|
| Protease | activity of enzymes in | enzymes in SGF-NP + | enzymes in SFG-NP + |
| Enzyme # | SGF-NP from Table 7, Col. C | 10% Ca Citrate | 0.3% Cr Polynicotinate |
| 1 + 4 | 0M | 11.5M* | 0.2M* |
| 1 + 5 | 0M | 6.6M* | 0.2M* |
| 4 + 5 | 0M | 12.2M* | 0.1M* |

TABLE 13
Proteolytic Activity of Protease Enzyme Combinations
in SGF-WP and Matrix Modifiers with CPC

| | Measured proteolytic | Proteolytic activity of | Proteolytic activity of |
|---|---|---|---|
| Protease | activity of enzymes in | enzymes in SGF-WP + | enzymes in SFG-WP + |
| Enzyme # | SGF-WP from Table 7, Col. E | 10% Ca Citrate | 0.3% Cr Polynicotinate |
| 1 + 4 | 6.7M | 12.6M* | 9.2M* |
| 1 + 5 | 2.1M | 8.3M* | 9.3M* |
| 4 + 5 | 0.9M | 9.1M* | 7.0M* |

The addition of 10% calcium citrate to SGF-NP increased proteolytic activity in all three protease enzyme combinations and with all three proteins tested. The addition of 0.3% chromium (III) polynicotinate to SGF-NP shows some increase in the Protease Enzyme #1+#5 combination with WPC, but the other protease enzyme combinations show no effect to a slight negative effect with WPC and SPC. However, a positive effect on proteolytic activity was reported with CPC for all three protease enzyme combinations, in the absence of pepsin, with the addition of both 10% calcium citrate and 0.3% chromium (III) polynicotinate (Tables 8, 10 and 12).

Results using SGF-WP containing 10% calcium citrate report an increase proteolytic activity with WPC, no effect to slight negative effect with SPC and an unexpected very positive effect with CPC substrate. The addition of 0.3% chromium (III) polynicotinate to SGF-WP is reported to show a decrease in proteolytic activity to approximately single enzyme rate with WPC substrate. A slight negative effect to no effect is reported with SPC substrate and an unexpected very positive effect on increased proteolytic activity is reported with CPC substrate (Tables 9, 11 and 13).

### CONCLUSION

The objective of this research was to determine the enzyme or enzyme combination that could most rapidly digest protein and promote protein absorption at conditions representing the environment of the gastrointestinal system. The results obtained were unexpected given the specifications and activity assays for each enzyme provided by the manufacturer. As an example, the manufacturer specifications indicate the average active pH and temperature range for Proteolytic Enzymes #1-45 is from pH 4.5 to 10.1 and 25-70° C. The optimal pH range is 7-8, and the optimal temperature range was 45-55° C. It would be expected that since their optimal pH and temperature range were very similar, and all experimental activities standardized to 7500 HUT/g protein, that Proteolytic Enzymes #1-#5 and the Reference enzyme blend would have very similar activity in SGF-NP with either WPC or SPC as substrates. However, as shown above, Proteolytic Enzymes #1-#6 and the Reference enzyme blend do not exhibit the same proteolytic activity with different proteins, in the presence and absence of pepsin and in combination. Further still, as evidenced by the tables above, Proteolytic Enzymes #1-#6, singly and in combination as indicated above, unexpectedly exhibit surprising proteolytic activity in the presence of pepsin or pepsin and a weak organic acid or polyphenolic compound, which has not been previously known or understood in the art.

While the disclosure has been described above with reference to specific embodiments thereof, it is apparent that many changes, modifications, and variations can be made without departing from the concept disclosed herein. Accordingly, it is intended to embrace all such changes, modifications, and variations that fall within the spirit and broad scope of the appended claims.

What is claimed is:

1. A food supplement comprising at least one protease enzyme selected from the group consisting of:
(i) CAS #9001-92-7; IUB 3.4.23.18 protease from Aspergillus:
(ii) CAS #9001-92-7, IUB 3.4.21.7 protease from Bacillus;
(iii) CAS #9001-61-0, IUB 3.4.11.1 protease from Aspergillus:
(iv) CAS #9074-07-1, IUB 3.4.21.63 protease from Aspergillus;
(v) CAS #9073-78-3, IUB 3.4.24.27 protease from Aspergillus;
(vi) CAS #9025-49-4, IUB 3.4.23.18 protease from Aspergillus: and
(vii) combinations thereof.

2. The food supplement of claim 1, wherein:
CAS #9001-92-7, IUB 3.4.23.18 protease from Aspergillus is from the species oryzae;
CAS #9001-92-7, IUB 3.4.21.7 protease from Bacillus is from the species subtilis;
CAS #9001-61-0, IUB 3.4.11.1 protease from Aspergillus is from the species oryzae;
CAS #9074-07-1, IUB 3.4.21.63 protease from Aspergillus is from the species melleus;
CAS #9073-78-3, IUB 3.4.24.27 protease from Aspergillus is from the species oryzae; and
CAS #9025-49-4, IUB 3.4.23.18 protease from Aspergillus is from the species niger.

3. The food supplement of claim 1, wherein the at least one protease enzyme is a combination of (i) CAS #9001-92-7, IUB 3.4.23.18 protease from Aspergillus and (iv) CAS #9074-07-1, IUB 3.4.21.63 protease from Aspergillus.

4. The food supplement of claim 1, wherein the at least one protease enzyme is a combination of (i) CAS #9001-92-7, IUB 3.4.23.18 protease from Aspergillus and (v) CAS #9073-78-3, IUB 3.4.24.27 protease from Aspergillus.

5. The food supplement of claim 1, wherein the at least one protease enzyme is a combination of (iv) CAS #9074-07-1, IUB 3.4.21.63 protease from Aspergillus and (v) CAS #9073-78-3, IUB 3.4.24.27 protease from Aspergillus.

6. The food supplement of claim 1, further comprising a protein.

7. The food supplement of claim 6, wherein the protein is selected from the group consisting of whey, soy, casein, and combinations thereof.

8. The food supplement of claim 1, further comprising an additional ingredient selected from the group consisting of a stabilizer, a matrix modifier, a carrier, a preservative, a bulking agent, a dessicant, an emulsifier, an enzyme coating, and combinations thereof.

9. The food supplement of claim 8, wherein the matrix modifier is selected from the group consisting of salts of dihydrogen phosphate, salts of nitric acid, salts of citric acid, salts of polynicotinic acid, salts of picolinic acid, weak organic acids, polyphenolic compounds with at least one pka of 5 or less, and combinations thereof.

10. The food supplement of claim 6, wherein the at least one protease enzyme is:
(i) CAS #9001-92-7, IUB 3.4.23.18 protease from Aspergillus oryzae;
(ii) CAS #9001-92-7, IUB 3.4.21.7 protease from Bacillus subtilis;
(iii) CAS #9001-61-0, IUB 3.4.11.1 protease from Aspergillus oryzae;
(iv) CAS #9074-07-1, IUB 3.4.21.63 protease from Aspergillus melleus; or
(v) CAS #9073-78-3, IUB 3.4.24.27 protease from Aspergillus oryzae; and
wherein the protein is whey or soy.

11. The food supplement of claim 3, further comprising a protein selected from the group consisting of whey, soy, casein, and combinations thereof,
wherein CAS #9001-92-7, IUB 3.4.23.18 protease from Aspergillus is from the species oryzae and CAS #9074-07-1, IUB 3.4.21.63 protease from Aspergillus is from the species melleus.

12. The food supplement of claim 11, further comprising a matrix modifier selected from the group consisting of calcium citrate and chromium (III) polynicotinate, and
wherein the protein is whey or casein.

13. The food supplement of claim 4, further comprising a protein selected from the group consisting of whey, soy, casein, and combinations thereof,
wherein CAS #9001-92-7, IUB 3.4.23.18 protease from Aspergillus is from the species oryzae and CAS #9073-78-3, IUB 3.4.24.27 protease from Aspergillus is from the species oryzae.

14. The food supplement of claim 13, wherein the protein is whey.

15. The food supplement of claim 13, further comprising a matrix modifier selected from the group consisting of calcium citrate and chromium (III) polynicotinate, and
wherein the protein is whey or casein.

16. The food supplement of claim 5, further comprising a protein selected from the group consisting of whey, soy, casein, and combinations thereof,
wherein CAS #9074-07-1, IUB 3.4.21.63 protease from Aspergillus is from the species melleus and CAS #9073-78-3, IUB 3.4.24.27 protease from Aspergillus is from the species oryzae.

17. The food supplement of claim 16, wherein the protein is whey.

18. The food supplement of claim 16, further comprising a matrix modifier selected from the group consisting of calcium citrate and chromium (III) polynicotinate.

19. A method of increasing protein absorption in the gastrointestinal system of a human being comprising the step of:
ingesting the food supplement of claim 1.

20. The method of claim 19, wherein protein absorption in the gastrointestinal system is increased in the presence of pepsin.

21. The method of claim 19, wherein the protein being absorbed is selected from the group consisting of whey, soy, casein, and combinations thereof.

22. The method of claim 19, wherein the at least one protease enzyme in the food supplement is a combination of (i) CAS #9001-92-7, IUB 3.4.23.18 protease from Aspergillus oryzae and (iv) CAS #9074-07-1, IUB 3.4.21.63 protease from Aspergillus melleus.

23. The method of claim 19, wherein the at least one protease enzyme in the food supplement is a combination of (i) CAS #9001-92-7, IUB 3.4.23.18 protease from Aspergillus oryzae and (v) CAS #9073-78-3, IUB 3.4.24.27 protease from Aspergillus oryzae.

24. The method of claim 19, wherein the at least one protease enzyme in the food supplement is a combination of (iv) CAS #9074-07-1, IUB 3.4.21.63 protease from Aspergillus melleus and (v) CAS #9073-78-3, IUB 3.4.24.27 protease from Aspergillus oryzae.

25. The method of claim 19, further comprising the step of ingesting protein.

26. The method of claim 25, wherein protein is ingested before, during or after the food supplement is ingested.

27. The method of claim 25, wherein the food supplement further comprises protein.
